(19) United States
(12) Patent Application Publication
(10) Pub. No.: US 2024/0036128 A1
(43) Pub. Date: Feb. 1, 2024

(54) SYSTEM AND APPARATUS FOR AWAKE FUNCTIONAL MRI OF AN ANIMAL SUBJECT

(71) Applicant: The General Hospital Corporation, Boston, MA (US)

(72) Inventors: Xin Yu, Malden, MA (US); Yuanyuan Jiang, Malden, MA (US); David C. Hike, Somerville, MA (US); Bei Zhang, Malden, MA (US); Zeping Xie, Malden, MA (US)

(21) Appl. No.: 18/363,256

(22) Filed: Aug. 1, 2023

Related U.S. Application Data
(60) Provisional application No. 63/394,029, filed on Aug. 1, 2022.

(51) Int. Cl. G01R 33/34; A61B 5/00; A61B 5/055; A61B 5/0205

(52) U.S. Cl. CPC G01R 33/34; A61B 5/0042; A61B 5/055; A61B 5/0205; A61B 5/6868; A61B 2503/40

(57) ABSTRACT

A system for awake functional magnetic resonance imaging (fMRI) of an animal subject includes an RF coil apparatus and a tunnel apparatus. The RF coil apparatus includes an RF coil configured to be implanted on the animal subject, a head post coupled to the RF coil and comprising a housing and a circuit board positioned within the housing, and a connector coupled to the circuit board. The tunnel apparatus includes a first end having an opening, a second end, a slot positioned on a top side of the tunnel apparatus and configured to movably receive the head post of the RF coil apparatus, and an inner region configured to receive the animal subject and allow the animal subject to move from the first end to the second end.

### CROSS-REFERENCE TO RELATED APPLICATIONS

This application is based on, claims priority to, and incorporates herein by reference in its entirety U.S. Ser. No. 63/394,029 filed Aug. 1, 2022, and entitled “Awake Animal Functional MRI Mapping Platform.”

### STATEMENT REGARDING FEDERALLY SPONSORED RESEARCH OR DEVELOPMENT

This invention was made with government support under award numbers NS124778 and NS121642 awarded by the National Institutes of Health. The government has certain rights in the invention.

### FIELD

The present disclosure relates generally to functional magnetic resonance (fMRI) systems and more particularly to a system and platform for awake fMRI of an animal subject.

### BACKGROUND

Preclinical fMRI has provided a unique functional mapping scheme for basic mechanistic and translational studies. In particular, animal fMRI enables brain-wide hemodynamic measurements, showing the non-invasive and longitudinal imaging capability, which is crucial for diagnostic and therapeutic testing in animal models. More importantly, preclinical fMRI can be combined with other imaging modalities, e.g., electrophysiological recordings or fiber photometry, to measure the brain dynamic signals across the neuro-glial-vascular (NGV) network. This multi-modal neuroimaging scheme can provide critical cross-scale information to identify bio-marker or therapeutic targets of a broad spectrum of brain disorders and lesions. An ongoing challenge of preclinical fMRI is how to reliably implement it in animal models with high throughput. Additional challenges of brain imaging of animal models or subjects include the susceptibility-related inhomogeneity (e.g., air-tissue interface distortion) that can affect image quality, signal sensitivity (e.g., achievement of cellular or micro-vascular specificity needs higher SNR for microscopic imaging), and limited space for a multi-modal environment setup.

### SUMMARY

In accordance with an embodiment, a system for awake functional magnetic resonance imaging (fMRI) of an animal subject includes an RF coil apparatus and a tunnel apparatus. The RF coil apparatus includes an RF coil configured to be implanted on the animal subject, a head post coupled to the RF coil and comprising a housing and a circuit board positioned within the housing, and a connector coupled to the circuit board. The tunnel apparatus includes a first end having an opening, a second end, a slot positioned on a top side of the tunnel apparatus and configured to movably receive the head post of the RF coil apparatus, and an inner region configured to receive the animal subject and allow the animal subject to move from the first end to the second end.

### BRIEF DESCRIPTION OF THE DRAWINGS

The present disclosure will hereafter be described with reference to the accompanying drawings, wherein like reference numerals denote like elements.

FIG. 1 shows a radio frequency (RF) coil apparatus for functional magnetic resonance imaging (fMRI) of an animal subject in accordance with an embodiment;

FIG. 2 shows a top view and a side view of an example single loop RF coil in accordance with an embodiment;

FIG. 3 shows a top view and a side view of an example figure eight RF coil in accordance with an embodiment;

FIG. 4 shows a top view and a side view of an example clover style RF coil in accordance with an embodiment;

FIG. 5 shows an example combined single loop and FIG. 8 loop RF coil in accordance with an embodiment;

FIG. 6 shows an example combined single loop and clover style RF coil in accordance with an embodiment;

FIG. 7 is a circuit diagram of an example RLC circuit for an RF coil apparatus in accordance with an embodiment;

FIG. 8 is a perspective view of a tunnel apparatus for fMRI of an animal subject in accordance with an embodiment;

FIG. 9 illustrates a tunnel apparatus positioned in an MM system in accordance with an embodiment;

FIG. 10 is a perspective view of an RF apparatus and the first end of a tunnel apparatus in accordance with an embodiment;

FIG. 11 is a perspective view an RF apparatus and a tunnel apparatus in accordance with an embodiment;

FIG. 12 is a perspective view of an RF apparatus and the second end of a tunnel apparatus in accordance with an embodiment; and

FIG. 13 is a front view of an RF apparatus and the second end of a tunnel apparatus in accordance with an embodiment.

### DETAILED DESCRIPTION

The present disclosure describes a system and apparatus for awake functional magnetic resonance imaging (fMRI) of an animal subject or animal model (e.g., a rodent, passerine, or small primate). In some embodiments, the system can include an implantable RF coil apparatus and a tunnel apparatus. The system may be configured to be used with a magnetic resonance imaging (MM) system (e.g., known MRI systems for imaging of animal subjects including preclinical MRI systems).to acquire magnetic resonance (MR) signals or data from the animal subject, for example, using pulse sequences for fMRI. In some embodiments, the acquired MR data may be used to generate fMRI images or maps. The RF coil apparatus may be used to receive radio frequency (RF) signals, to transmit RF signals, or to both receive and transmit RF signals. The tunnel apparatus may be configured to provide an awake animal head-fixation device for the animal subject and to allow the animal subject to run through the tunnel apparatus with a head-fixed posture and to fix the head position of the animal subject for imaging, for example, brain imaging.

In some embodiments, the RF coil apparatus includes an implantable RF coil and a circuit board coupled to the implantable RF coil. The implantable RF coil may be mounted directly on the skull (and brain) (or inserted into the craniotomy, i.e., a large opening) of the animal subject. The circuit board (e.g., a printed circuit board (PCB)) may have a cover or housing and can be used as a head post when positioned in the tunnel apparatus and can be used to restrain the animal subject in a head-fixed position. In some embodiments, the circuit board can include an RLC circuit which can be adjusted for various applications. In some embodiments, the RF coil apparatus can also include a connector coupled to the circuit board. The tunnel apparatus can include a slot or track that may be configured to receive the circuit board (or head post) of the RF coil apparatus to allow an animal subject with the implanted RF coil apparatus to move voluntarily (or passively) through the tunnel apparatus with a head-fixed posture. In some embodiments, the tunnel apparatus allows the animal subject to move through the tunnel apparatus to an area where the head post can be fixed to the slot or track (e.g., a head fixation point). In some embodiment, when the tunnel apparatus is positioned in a bore of a MM system, the head fixation point may correspond to a geometric center of the MRI system for imaging. In some embodiments, the tunnel apparatus may include an attachment or connection mechanism which may be used to removable attach a cassette (e.g., a miniaturized cassette) that may be configured to include a preamplifier and, in some embodiments, a transmit/receive switch. The cassette may be connected to the connector of the RF coil apparatus.

The disclosed system for awake functional magnetic resonance imaging (fMRI) of an animal subject advantageously can improve image quality, provide a superior signal-to-noise ratio (SNR) measurement, and improve ease of use for acquiring images (e.g., brain images) of an animal subject. To address the physiological confound, the system can advantageously be implemented for imaging without anesthetic treatment for the animal subject. Because the RF coil of the apparatus is mounted directly on the skull of the animal subject, there is less motion-induced loading change, which significantly reduces the B1 variability. In some embodiments, no additional tuning or matching is needed once an adjustable capacitor (e.g., a trimmer) of the circuit board of the RF coil apparatus is set with appropriate values for an application. The geometry and mounting scheme of the RF coil apparatus advantageously reduces air-tissue B0 and motion-related loading-based B1 artifacts. The tunnel apparatus which can include a slot to movably (or slidably) receive the head post (i.e., the circuit board and housing) of the RF coil apparatus allows an animal subject to move through an inner chamber of the runnel apparatus and can advantageously reduce the stress of the animal subject(s) and enable behavioral testing of the animal subject(s). The miniaturized removable (e.g., plug-and-play) cassette (e.g., holding a preamplifier and TR switch) can improve the SNR of images generated using the MR data acquired with the RF coil apparatus. In some embodiments, the design and loading characteristics of the RF coil apparatus can be designed specifically for a designed application. For example, the RF coil apparatus may be designed for specific species application (e.g., rodent, passerine, and small primate). In some embodiments, the disclosed system for awake functional magnetic resonance imaging (fMRI) of an animal subject can allow multi-modal schemes, for example, the system can be combined with a miniaturized camera (or cameras), glass/coverslip plug implantation for optical imaging, fiber photometry, or electrophysiological recordings.

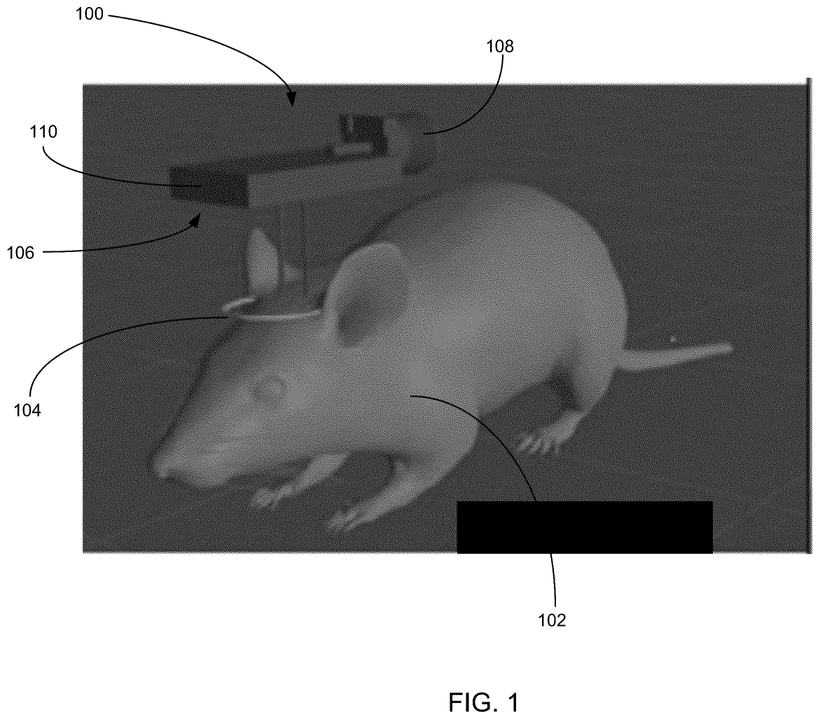

As mentioned above, in some embodiments, the system for awake fMRI of an animal subject (or animal model) can include an RF coil apparatus and a tunnel apparatus. FIG. 1 shows a radio frequency (RF) coil apparatus for functional magnetic resonance imaging (fMRI) of an animal subject in accordance with an embodiment. The RF coil apparatus 100 advantageously can reduce air-tissue B0 and motion-related loading-based B1 artifacts. In FIG. 1, an RF coil apparatus 100 can include an RF coil 104, a circuit board/head post 106, and a connector 108. The RF coil 104 is implantable and may be mounted to an animal subject 102 (e.g., a rodent, passerine, or small primate) as illustrated in FIG. 1. While the animal subject 102 in FIG. 1 (as well as in FIGS. 10, 11 and 13 discussed below) is illustrated as a rodent, it should be understood that the disclosed system for awake fMRI may be used with other animal subjects such as, for example, passerine, small primate, etc. The RF coil is coupled and connected to the circuit board/head post 106. The circuit board/head post 106 may include a housing or cover 110. The connector 108 is coupled to and connected to the circuit board 106. As discussed above, the RF coil apparatus 100 may be used for various fMRI applications, for example, for head-fixed brain imaging of an animal subject.

In some embodiments, the RF coil 104 may advantageously be directly mounted to the skull (and brain) of the animal subject 102. Accordingly, the RF coil 104 may be implanted on the head of the animal subject 102. In some embodiments, the implanted RF coil can be fixed using a material such as, for example, dental cement. Minimizing the distance of the RF coil to the skull can produce an idealized loading scheme and improve B1 sensitivity. In some embodiments, the RF coil 104 may be formed from, for example, magnet wire (e.g., copper wire coated with a thin layer of enameled insulation. In some embodiments, the RF coil 104 may be formed using standard copper wire or be printed on a PCB as copper layers. In some embodiments, the RF coil 104 may be formed from a flexible circuit mounted to a rigid material (e.g., dense rubber or other nonconductive polymer) to ensure proper mounting for head fixation. The size and loading characteristics of the RF coil 104 may be designed specifically for the imaging application desired by an operator or end user. This can allow for designs of the RF coil 104 to be built for specific species applications such as, for example, rodent, passerine, and small primate. The overall weight of the RF coil apparatus 100 may be dependent on the size and shape of the RF coil 104. In some embodiments, the overall weight of the RF coil apparatus 100 may be in the range of 2.0 g to 2.5 g which can allow for implantation in smaller animal subjects (e.g., mice). In some embodiments, the size of the RF coil 104 may be in the range of 0.5 mm to mm, which can allow for coverage of the whole brain of the animal subject 102, as well as, for example, direct implantation above the dura on the skull through craniotomy. Animal subjects with RF coil implantations can have a high durability and attachment longevity, which can be maintained, for example, for over six weeks.

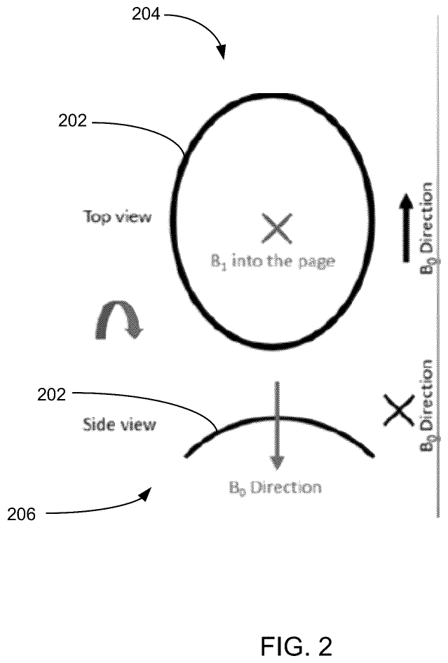

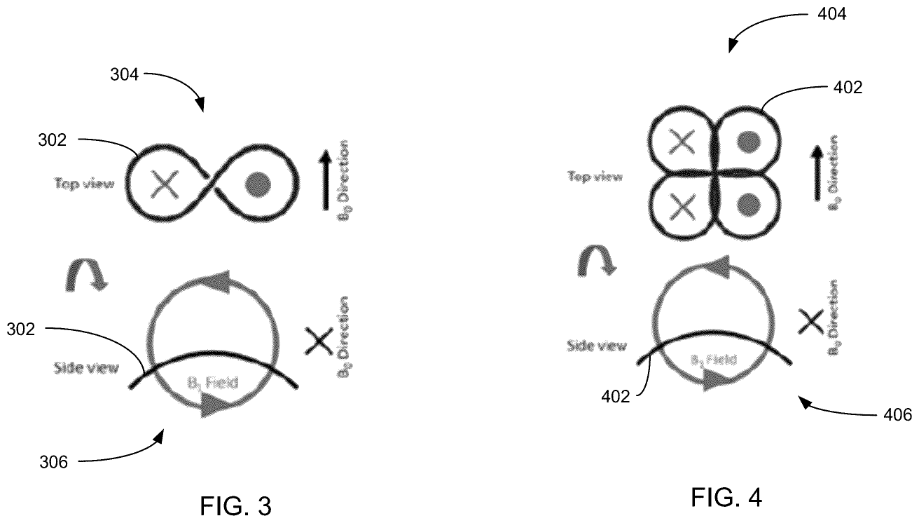

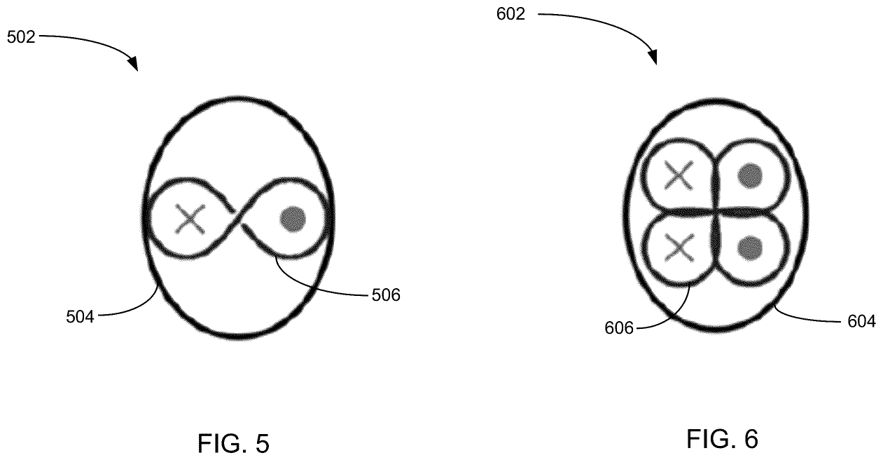

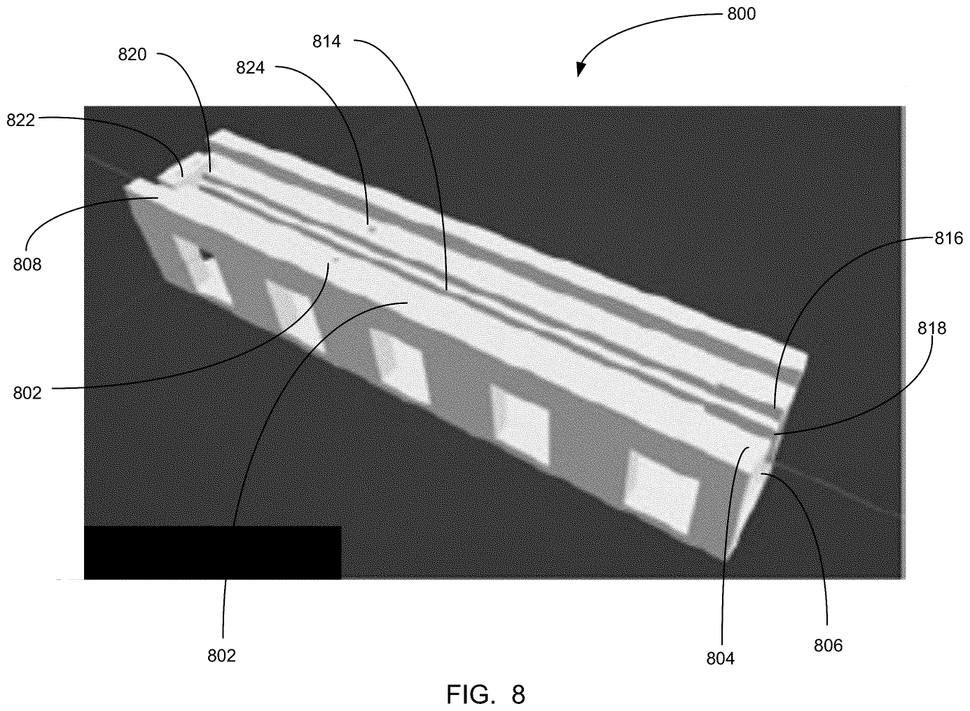

In some embodiments, various coil or loop shapes can be used for RF coil 104 to provide, for example, a single channel transceiver. For example, in some embodiments, the RF coil 104 may be implemented as a single loop coil 202 as shown in FIG. 2, a FIG. 8 loop coil 302 as shown in FIG. 3, or a “clover” shaped loop coil 402 as shown in FIG. 4. In FIG. 2, a top view 204 and a side view 206 of the example single loop coil 202 are shown. In FIG. 2 the “X” inside a loop represents B1 into the page. In FIG. 3, a top view 304 and a side view 306 of an example FIG. 8 loop coil 302 are shown. In FIG. 4, a top view 404 and side view 406 of an example “clover” shaped loop coil 402 are shown. In FIGS. 3 and 4, the “X” inside a loop represents B1 into the page and the circle “*” inside a loop represents B1 out of the page for the respective coil. In some embodiments, the different B1 directions of multiple coils shapes can be combined for a multi-array coil design. FIG. 5 shows an example combined single loop and FIG. 8 loop RF coil in accordance with an embodiment. In FIG. 5, the RF coil 502 includes a single loop 504 combined with a FIG. 8 loop 506. The different B1 directions of the single loop 502 and FIG. 8 loop 506 can enable parallel acquisition. FIG. 6 shows an example combined single loop and clover style loop RF coil in accordance with an embodiment. In FIG. 6, the RF coil 602 includes a single loop 604 combined with a clover style loop 606. In FIGS. 5 and 6, the “X” inside a loop represents B1 into the page and the circle “*” inside a loop represents B1 out of the page for the respective coil.

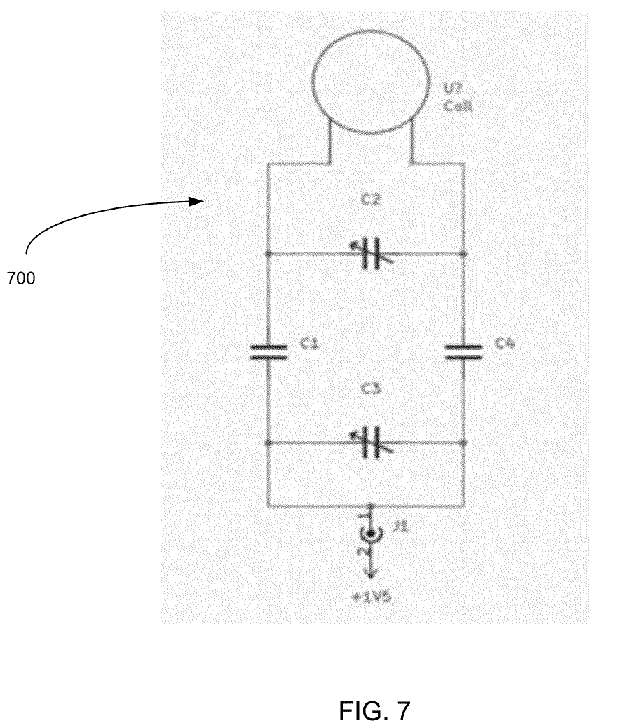

Returning to FIG. 1, the circuit board 106 of the RF coil apparatus 100 can be positioned in a housing or cover 110 and may be used as a head post to retrain the animal subject 102 in a head-fixed position. In some embodiments, when the animal subject is not being imaged, the housing or cover can be configured to isolate and protect the circuit from collisions with the housing in which the animal subject is kept or the feeding equipment for the animal subject, or to protect the circuit from animal bedding falling onto the connector 108. In some embodiments, the circuit board 106 may include an RLC circuit that may be adjusted for various applications. FIG. 7 is a circuit diagram of an example RLC circuit 700 for an RF coil apparatus in accordance with an embodiment. In some embodiments, the frequency of the RLC circuit 700 can be adjusted to work with any MR-compatible nuclei (e.g., 1H, 23Na, 13C, etc.) over a wide range of magnetic field strengths (e.g., 4.7 T-21 T). A simple design for the circuit of the circuit board 106 shown in FIG. 1), e.g., RLC circuit 700, can help minimize the weight of the RF coil apparatus 100 (shown in FIG. 1) when mounted to, for example, the head of an animal subject. Another advantage of the design of the circuit board 106 is that, in some embodiments, the tuning a matching trimmer capacitors may be soldered on the circuit board in a tight configuration to minimize the space used and reduce the overall size and weight of RF coil apparatus 100 while still allowing the RF coil 104 to be easily tuned/matched by an operator or end user. Overall weight reduction of the RF coil apparatus 100 can advantageously allow for smaller animal subjects (e.g., mice) to be kept and imaged over long time courses. In some embodiments, the circuit board 106 may be configured to be removably attached to the implantable RF coil 104 to further remove the weight of the circuit board 106 when the animal subject is not being imaged.

The connector 108 of the RF coil apparatus 100 is coupled to the circuit board 106 and housing 110. In some embodiments, the connector may be, for example, a micro coaxial (MCX) connector, a subMiniature version A (SMA) connector, or a subMiniature version B (SMB) connector. In some embodiments, the connector 108 can be further customized to minimize or eliminate the need for plug adapters which can cause disruptions resulting in poor signal transmission.

FIG. 8 is a perspective view of a tunnel apparatus for fMRI of an animal subject in accordance with an embodiment. The tunnel apparatus 800 is configured to allow an animal subject (not shown) with the RF coil apparatus 100, 840 (as described above with respect to FIG. 1 and also shown in FIGS. 10-13) move through a chamber 810 inside the tunnel apparatus 800 in a head-fixed posture. Advantageously, the tunnel apparatus 800 can significantly reduce the stress of the animal subject and enable behavioral testing. The tunnel apparatus 800 can include a top side 802, a first end 804, a second end 808 and a chamber or inner region 810. In some embodiments, as illustrated in FIGS. 8-13, the tunnel apparatus 800 can have a box or rectangular shape. The chamber can be configured to accommodate different species of animal subject by size and standing posture.

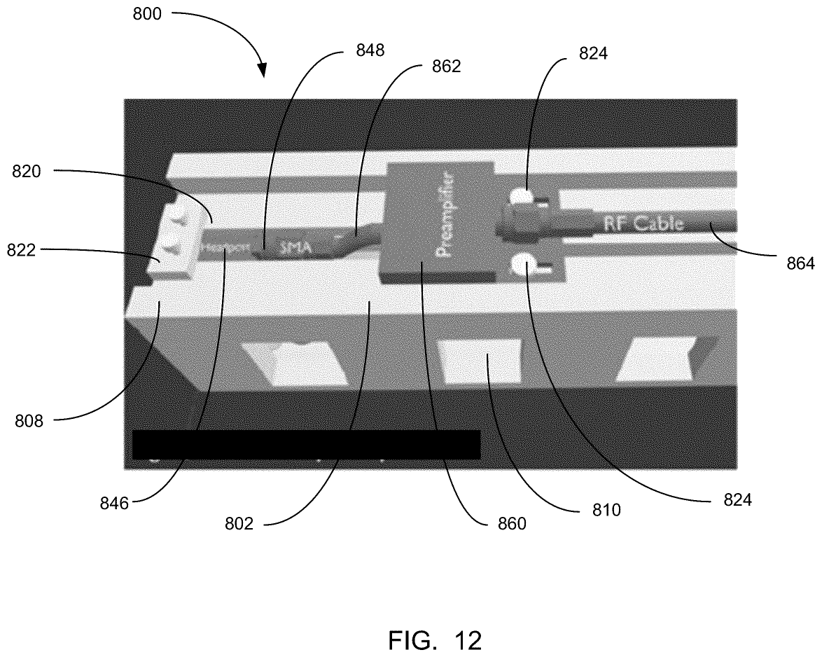

The first end 804 of the tunnel apparatus 800 can include an opening or entrance 806 (also shown in FIG. 10) that is configured to allow an animal subject with an implanted RF coil apparatus (e.g., RF coil apparatus 100 shown in FIG. 1) to enter the inner region 810 of the tunnel apparatus. The top side 802 of the tunnel apparatus 800 can include a slot (or track) 814 that is configured to receive the circuit board/head post (e.g., circuit board/head post 106 shown in FIG. 1) of the implantable RF coil apparatus. The slot (or track) 814 can have a first end 816 corresponding to the first end 804 of the tunnel apparatus 800 and a second end 820 corresponding to the second end 808 of the tunnel apparatus 800. The first end 816 of the slit (or track) 814 can have an opening 818 to configured to receive the circuit board/headpost of the implantable RF coil apparatus. The second end 820 of the slot (or track) 814) is closed, for example, using a stop or end piece 822. The top side 802 of the tunnel apparatus 800 may also include one or more attached or connection mechanisms 824 for attaching, for example, a cassette (e.g., cassette 860, shown in FIG. 12) containing a preamplifier and a transmit/receive (T/R) switch and/or a transmit coil (both of which are discussed further below with respect to FIG. 12).

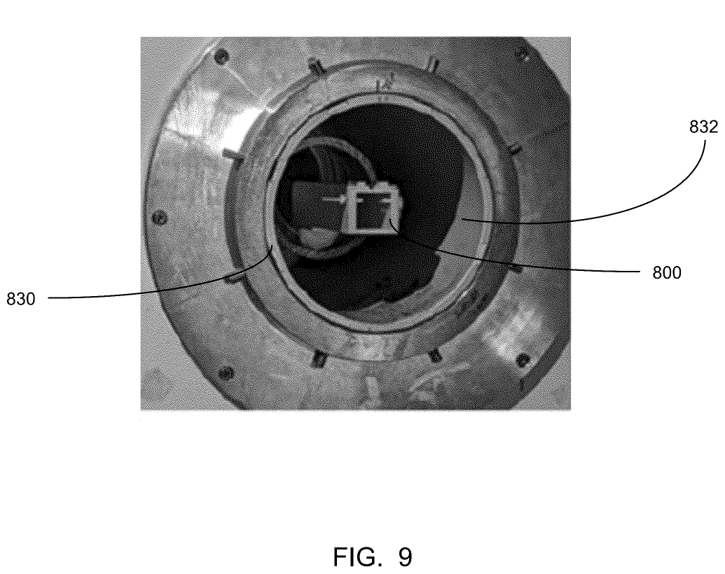

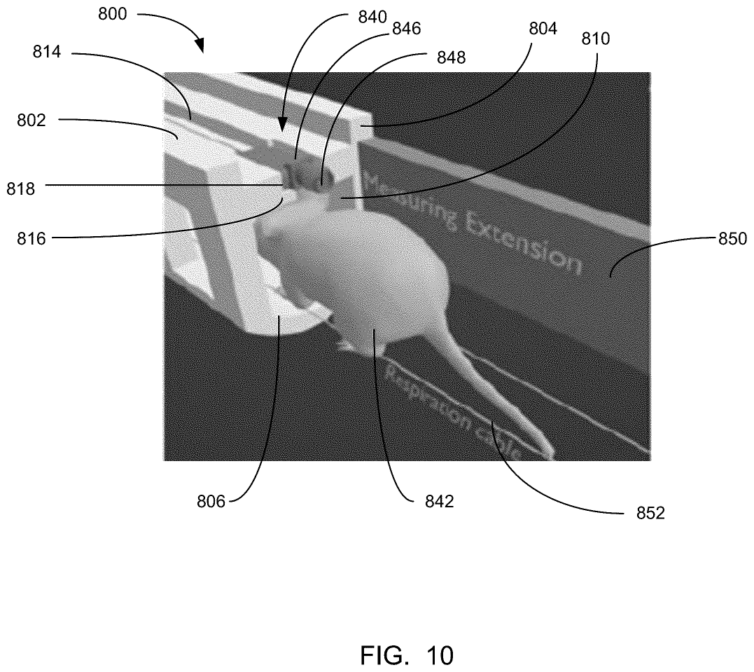

In some embodiments, the size and shape of the tunnel apparatus 800 can be configured based on the size of a bore (i.e., as defined by the gradient size) of an MM system in which the tunnel apparatus 800 may be positioned for imaging of the animal subject. In some embodiments, the tunnel apparatus 800 may be configured for use with gradient bore sizes ranging from 6 cm to 21 cm. FIG. 9 illustrates a tunnel apparatus positioned in an MM system in accordance with an embodiment. In the example shown in FIG. 9. the tunnel apparatus 800 is shown positioned in an example MRI system with a gradient 830 and a bore 832 having a size of 12 cm. In some embodiments, once an animal subject has been positioned in the tunnel apparatus 800 and fixed at a desired head fixation point (e.g., at the second end 820 of the slot/track 814), the tunnel apparatus 800 may be positioned in the bore 832 of the MRI system (or scanner) for imaging using, for example, known fMRI acquisition techniques.

As mentioned, an animal subject may move through the inner region (or chamber) 810 of the tunnel apparatus 800 from the first end 804 to the second end 808. FIG. 10 is a perspective view of an RF apparatus and the first end of a tunnel apparatus in accordance with an embodiment. As shown in FIG. 10, the first end 804 of the tunnel apparatus 800 can include an opening (or entrance) 806 that is configured to allow an animal subject 842 with an implanted RF coil apparatus 840 (e.g., RF coil apparatus 100 shown in FIG. 1) to enter the inner region 810 of the tunnel apparatus 800. The circuit board/head post 846 of the RF coil apparatus 840 can be positioned in the slot (or track) 814 on the top side 802 of the tunnel apparatus 800 (e.g., via the opening 818 in the first end 816 of the slot (or track) 814). The circuit board/head post 814 may be configured to slide in the slot (or track) 814 as the animal subject moves through the inner region 810 toward the second end 808 (not shown in FIG. 10) of the tunnel apparatus 800. In some embodiments, the tunnel apparatus 800 may also include a measurement extension 850 (e.g., located on a side of the tunnel apparatus 800) which can be used to locate the RF coil apparatus 840 to the center of the gradient (e.g., gradient 830 shown in FIG. 9) of an MM system when the tunnel apparatus 800 is positioned in the bore (e.g., bore 832 shown in FIG. 9) of the MRI system. For example, in some embodiments, the measurement extension 850 may be configured to measure the depth of the RF coil apparatus 840 in the bore of the MM system.

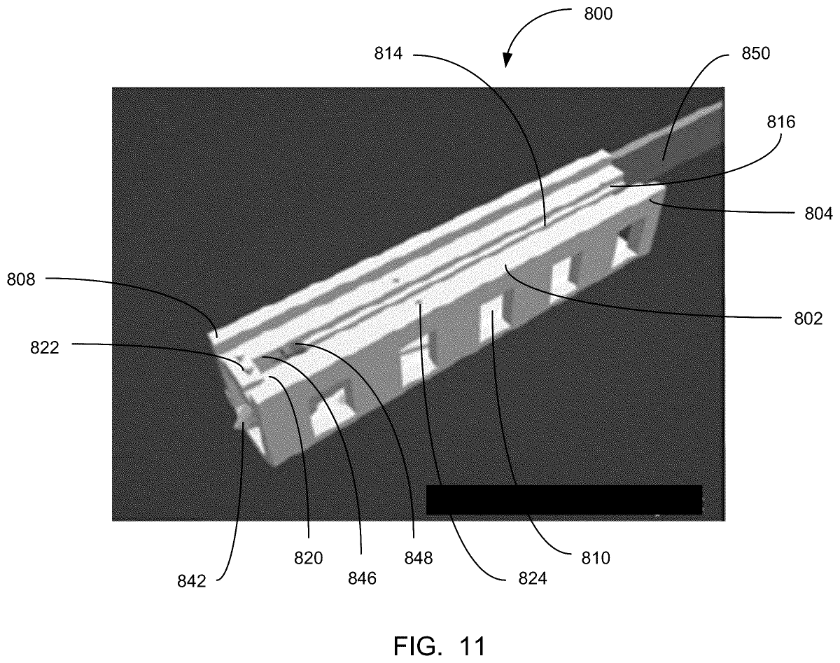

The second end 808 of the tunnel apparatus 800 may be configured to allow the circuit board/head post 846 of the RF coil apparatus 840 on the animal subject 842 to be fixed to the slot (or track) 814. FIG. 11 is a perspective view an RF apparatus and a tunnel apparatus in accordance with an embodiment. In FIG. 11, the animal subject 842 is shown at a position at the second end 808 of the tunnel apparatus 800. The animal subject 846 may be trained to move through the inner region or chamber 810 of the tunnel apparatus 800, in particular, to move from the first end 804 to the second end 808 while the circuit board/head post 846 of the RF coil apparatus 840 slides through the slot (or track) 814. For example, an animal subject 842 with the implanted RF coil apparatus 840 can learn to move voluntarily through the inner region 810 of the tunnel apparatus 800. In some embodiments, when the tunnel apparatus is positioned in a bore of an MRI system the second end 808 of the tunnel apparatus 800 corresponds to a geometric center of the MM system. The animal subject 842 can move through the inner region 810 of the tunnel apparatus 800 with a head-fixed posture. Advantageously, the system for awake fMRI of an animal subject can be implemented without anesthetic treatment for the animal subject 432. In some embodiments, the system can enable the imaging of multiple awake animal subjects when, for example, performing communicative exploratory behavior testing.

As mentioned, the circuit board 846 of the RF coil apparatus 800 can be used as a head post to restrain the animal subject in a head-fixed position. FIG. 12 is a perspective view of an RF apparatus and the second end of a tunnel apparatus in accordance with an embodiment. In FIG. 12, the animal subject (not shown) and the RF coil apparatus are positioned at the second end 808 of the tunnel apparatus 800 (and the second end 820 of the slot 814) after the animal subject has moved through the inner region (or chamber) 810 with the circuit board/head post 846 sliding through the slot (or track) 814. The second end 820 of the slot 814 (corresponding to the second end of the chamber 810) can be closed and can be configured as a head fixation point for the animal subject. In some embodiments, the circuit board/head post 846 may be manually fixed toward the second end 808 of the tunnel apparatus 800 and the second end 820 of the slot (or track) 814. In some embodiments, the circuit board/head post 846 can be driven by automatic control with an optical sensor mounted inside the bore (e.g., bore 832 shown in FIG. 9) of the MRI system.

In some embodiments, a cassette 860 (e.g., a miniaturized cassette) may be used to house a preamplifier and transmit/receive (T/R) switch that can be attached to the tunnel apparatus 800 and connected to the circuit board/head post 846 (and the RF coil, not shown) via the connector 848. As mentioned above, the connector 848 of the RF coil apparatus can be customized for different needs and applications. The RF coil apparatus 840 can be connected to an MM system via the preamplifier in the cassette 860 (e.g., using an RF cable 864) to ensure efficient and significantly improved SNR. The cassette 860 can be configured to be attached to the tunnel apparatus 800, for example, the cassette 860 may be attached to the tunnel apparatus 800 using one or more attachment or connection mechanisms 824 on the top side 802 of the tunnel apparatus 800. In some embodiments, the cassette 860 (e.g., a preamplifier with an integrated T/R switch) may be directly connected (or plugged into) the RF coil apparatus circuit board/head post 846 using the connector 848 and, for example, a cable 862 of the cassette 860. In some embodiments, when attached to the tunnel apparatus 800 and connected to the connector 848 of the circuit board/head post 846 of the implanted RF coil apparatus, the cassette 860 can also serve to fix the circuit board/head post 846 to the tunnel apparatus 800 to eliminate the longitudinal motion of the animal subject (not shown). In some embodiments, a separate transmit coil (e.g., a double-ring (Helmholtz-style) transmit coil) may be positioned laterally across the animal subject's head on the top side 802 of the tunnel apparatus 800. The implanted RF coil (not shown) can then act as a receive only coil which can eliminate the need to include a T/R switch in the preamplifier circuit block of the cassette 860.

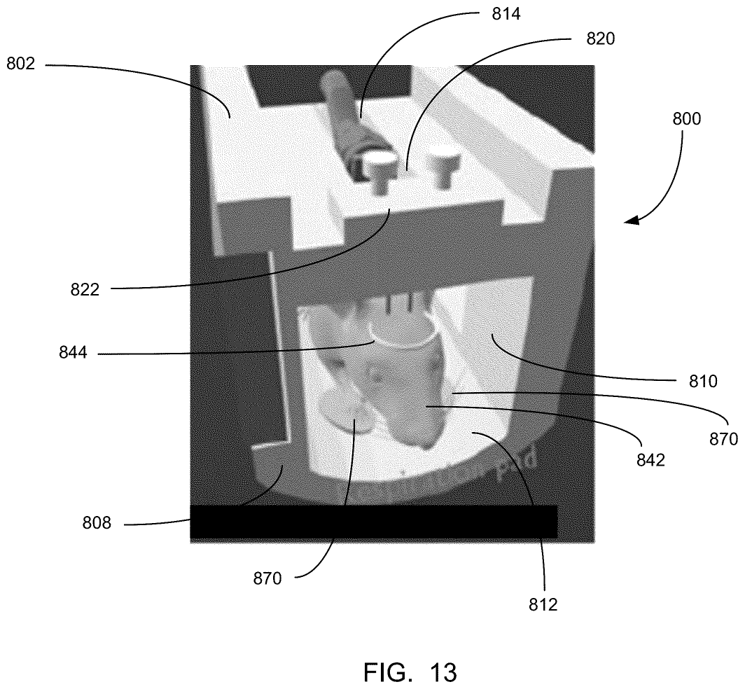

FIG. 13 is a front view of an RF apparatus and the second end of a tunnel apparatus in accordance with an embodiment. In FIG. 13, the animal subject 842 with an implanted RF coil 844 of the RF coil apparatus is shown in a fixed position at the second end 808 of the tunnel apparatus 800 and the second end 820 of the slot (or track) 814. In some embodiments, one or sensors 870 (e.g., pad sensors such as body contact pads, etc.) may be positioned on and attached to a bottom surface 812 of the inner region 810 of the tunnel apparatus 800. The sensor(s) 870 may be used to sense and acquire physiological data of the awake animal subject 842, for example, respiratory and electrocardiogram (ECG) signals, during scanning (e.g., for brain imaging). In some embodiments, the sensor(s) 870 may be used to acquire respiratory and ECG measurements of the animal subject 842 simultaneously with the MR signals. In some embodiments, the sensor(s) may be coupled to a cable 852 (shown in FIG. 10) that can be run along the bottom surface 812 of the inner region (or chamber) 810 of the tunnel apparatus 800.

In some embodiments, the system for awake fMRI of an animal subject can be incorporated with multi-modal brain imaging sets, such as, for example, miniaturized microscopy, fiber bundle imaging, and single/multichannel fiber photometry, which can simultaneously detect cell-type specific dynamics from neurons and astrocytes. In some embodiments, to provide multi-model schemes, the system for awake fMRI of an animal subject can be customized to include additional elements such as, for example, a miniaturized camera set (e.g., including an GRINS lens or multi-bundle fibers), as well as optogenetic stimulation fibers, electrodes, and microinjection capillaries. In some embodiments, the system for awake fMRI of an animal subject used in a multi-modal application, can allow longitudinal recording of cross-scale brain dynamic signals by expressing genetically encoded indicators for Calcium, Glutamate, or neuromodulators, and voltage sensors.

In some embodiments, awake fMRI may be used to study the cross-scale brain function across a large pool of transgenic mouse lines. In some embodiments, fMRI images generated from MR data (or signals) acquired using the disclosed system for awake fMRI of an animal subject can be denoised using various methods such as MPPCA (Marcenko-Pastur PCA), VST, 1-D wavelet, and multivariate-wavelet. In some embodiments, the disclosed system for awake fMRI of an animal subject can be used for high resolution (e.g., 100 μm) echo planar imaging (EPI) scan for brain imaging. In some embodiments, the system for awake fMRI of an animal subject can provide a higher SNR that can be at least two times better than a commercial 4-array coil (>40K).

The present invention has been described in terms of one or more preferred embodiments, and it should be appreciated that many equivalents, alternatives, variations, and modifications, aside from those expressly stated, are possible and within the scope of the invention.

1. A system for awake functional magnetic resonance imaging (fMRI) of an animal subject, the system comprising:
an RF coil apparatus comprising:
an RF coil configured to be implanted on the animal subject;
a head post coupled to the RF coil and comprising a housing and a circuit board positioned within the housing; and
a connector coupled to the circuit board; and
a tunnel apparatus comprising:
a first end having an opening;
a second end;
a slot positioned on a top side of the tunnel apparatus and configured to movably receive the head post of the RF coil apparatus; and
an inner region configured to receive the animal subject and allow the animal subject to move from the first end to the second end.

2. The system according to claim 1, further comprising a cassette removably attached to the top side of the tunnel apparatus at the second end of the tunnel apparatus and connected to the connector of the RF coil apparatus, wherein the cassette comprises a preamplifier.

3. The system according to claim 2, wherein the cassette further comprises a transmit/receive (T/R) switch.

4. The system according to claim 1, wherein the tunnel apparatus has a set of dimensions and the set of dimensions are configured to allow the tunnel apparatus to be positioned in a bore of an MRI system.

5. The system according to claim 1, wherein the RF coil apparatus is configured to receive magnetic resonance (MR) signals from the animal subject using the RF coil.

6. The system according to claim 1, wherein the RF coil apparatus is configured to transmit RF signals to the animal subject using the RF coil.

7. The system according to claim 1, wherein the RF coil is one of a single loop, a FIG. 8 loop or a clover style loop.

8. The system according to claim 1, wherein the RF coil comprises a single loop and a FIG. 8 loop.

9. The system according to claim 1, wherein the RF coil comprises a single loop and a clover style loop.

10. The system according to claim 1, wherein the tunnel apparatus further comprises at least one sensor positioned at the second end on a bottom surface of the inner region, wherein the at least one sensor is configured to measure physiological data of the animal subject.

11. The system according to claim 10, wherein the physiological data is one or more of respiratory data or electrocardiogram (ECG) data.

12. The system according to claim 10, wherein the at least one sensor is configured to acquire the physiological data simultaneously with MR data.

13. The system according to claim 1, wherein the head post is configured to slide along the slot on the top side of the tunnel apparatus between the first end and the second end.

14. The system according to claim 1, wherein the connector is one of a micro coaxial (MCX) connector, a subMiniature version A (SMA) connector, or a subMiniature version B (SMB) connector.

15. The system according to claim 1, wherein the RF coil is configured to be implanted on a head of the animal subject.

16. The system according to claim 1, wherein the circuit board includes an adjustable RLC circuit.
